SEQUENCE LISTING (4 sequences)

SEQ ID NO: 1 - DNA, 558 bp, Brassica napus
  source 1..558: mol_type = unassigned DNA
  misc_feature 1..558: note = IND-A1 coding sequence
  CDS 1..558: note = translation of CDS is given by Seq-ID 2; protein_id = 2; translation = MSGSKADAAIAPIVMMEHHHLLMNWNKPIDLITEENSFNHNPHF IVDPPSETLSHFQPPPTIFSDHGGGEEAEEEEEEEGEEEMDPMKKMQYAIAAMQPVDL DPATVPKPNRRNVRVSDDPQTVVARRRRERISEKIRILKRMVPGGAKMDTASMLDEAI RYTKFLKRQVRLASSASHSAWSSYV
atgtctggctcaaaagcagatgcagccatagccccaatagtcatgatggagcatcatcatctccttatgaattggaacaaacctattgatctcattacagaagaaaactcttttaaccacaatcctcatttcatagtagatccaccttccgaaaccctaagccacttccagcccccgccgacaatcttctccgatcacggaggaggagaggaagcagaagaagaagaagaagaagaaggagaggaagagatggatccgatgaagaagatgcaatacgcgattgctgccatgcagcccgtagacctcgatccagccaccgttcctaagccgaaccgccgtaacgtaagggtaagcgacgaccctcagacggtggtggctcgtcggcgtagagaaaggataagcgagaagatccggatattgaagaggatggtgccaggcggtgcaaagatggacactgcctccatgctcgacgaagccatccgctacaccaagttcttgaaacggcaggtgaggctagcttcttcagcctcacactcagcttggagctcctatgtctga

SEQ ID NO: 2 - protein, 185 aa, Brassica napus
  REGION 1..185: note = IND-A1 protein sequence
MSGSKADAAIAPIVMMEHHHLLMNWNKPIDLITEENSFNHNPHFIVDPPSETLSHFQPPPTIFSDHGGGEEAEEEEEEEGEEEMDPMKKMQYAIAAMQPVDLDPATVPKPNRRNVRVSDDPQTVVARRRRERISEKIRILKRMVPGGAKMDTASMLDEAIRYTKFLKRQVRLASSASHSAWSSYV

SEQ ID NO: 3 - DNA, 633 bp, Brassica napus
  source 1..633: mol_type = unassigned DNA
  misc_feature 1..633: note = IND-C1 coding sequence
  CDS 1..633: note = translation of CDS is given by Seq-ID 4; protein_id = 4; translation = MYKRKVYASLVQKLYMSGSKADAAAIAPIVMMEPHHLLMNWNKP IDLITQENSFNHNPHFMVDPPSETLSHFQPPPTVFSDPGGGEEAEDEEGEEEIDEMKE MQYAIAAMQPVDIDPATVPKPNRRNVRVSEDPQTVVARRRRERISEKIRILKRMVPGG AKMDTASMLDEAIRYTKFLKRQVRLLQPHTQLGAPMSDPSRLCYYHNSDT
atgtataaaagaaaggtctatgcgtctctagtccaaaaactctatatgtctggttcaaaagcagatgcagcagccatagccccaatagtcatgatggagcctcatcatctccttatgaactggaacaaacctattgatctcattacacaagaaaactcttttaaccacaatcctcatttcatggtagatccaccttccgaaaccctaagccacttccagcccccgccgacagtcttctccgatcccggaggaggagaggaagcagaagacgaagaaggagaggaagagatagatgagatgaaggagatgcaatacgcgattgctgccatgcagcccgtagacatcgatccagccaccgttcctaagccgaaccgccgtaacgtaagggtaagcgaggacccccagacggtggtggctcgtcggcgtagagaaaggataagcgagaagatccggatattgaagaggatggtgccaggcggtgcaaagatggacactgcctccatgcttgacgaagccatccgctacaccaagttcttgaaacggcaggtgaggcttcttcagcctcacactcagcttggggctcctatgtctgacccttctcgcctttgttattaccacaactcggatacctaa

SEQ ID NO: 4 - protein, 210 aa, Brassica napus
  REGION 1..210: note = IND-C1 protein sequence
MYKRKVYASLVQKLYMSGSKADAAAIAPIVMMEPHHLLMNWNKPIDLITQENSFNHNPHFMVDPPSETLSHFQPPPTVFSDPGGGEEAEDEEGEEEIDEMKEMQYAIAAMQPVDIDPATVPKPNRRNVRVSEDPQTVVARRRRERISEKIRILKRMVPGGAKMDTASMLDEAIRYTKFLKRQVRLLQPHTQLGAPMSDPSRLCYYHNSDT

(19) United States
(12) Patent Application Publication
(10) Pub. No.: US 2024/0381825 A1
(43) Pub. Date: Nov. 21, 2024

(54) METHOD FOR ENHANCING OIL CHARACTERISTICS IN BRASSICA OILSEEDS

(71) Applicant: BASF AGRICULTURAL SOLUTIONS SEED US LLC, Florham Park, NJ (US)

(72) Inventors: Jeffrey MANSIERE, Saskatoon (CA); Stewart BRANDT, Saskatoon (CA)

(21) Appl. No.: 18/688,934

(22) PCT Filed: Sep. 1, 2022
(86) PCT No.: PCT/US2022/075818

§ 371 Date: Mar. 4, 2024

(30) Foreign Application Priority Data
Sep. 3, 2021 (EP) 21194750.2

(51) Int. Cl. A01H 6/20; A01D 45/00; A01H 1/00; A01H 5/10; C12N 15/82

(52) U.S. Cl. CPC A01H 6/202; A01D 45/00; A01H 1/1205; A01H 5/10; C12N 15/8266

(57) ABSTRACT

The invention provides methods for enhancing oil characteristics in Brassica oilseed plants by growing said plants and harvesting seed from said plants by straight cutting. The harvested seeds are useful for the production of oil with enhanced characteristics which can be used, for example, as food ingredient.

### FIELD OF THE INVENTION

This invention relates to methods for enhancing oil characteristics, in particular oil quantity and reducing levels of saturated fatty acids, in Brassica oilseed seeds.

### BACKGROUND OF THE INVENTION

Members of the Brassica genus are economically important crops in particular for oil production in many countries of the world. Examples are Brassica napus, in particular spring oilseed rape, or canola, or winter oilseed rape, or Brassica juncea.

Important targets for Brassica oilseeds breeding are oil quantity and oil quality. Oil quality characteristics are improved to improve tastes, healthiness and performance.

Interest in reducing the levels of glucosinolates in seed results from the presence of bitter-tasting, toxic and goitrogenic degradation products which limit the incorporation of rape meal into non-ruminant animal feed.

The degree and/or amount of polyunsaturated fatty acids of vegetable oils are characteristic and determinative properties with respect to oil uses in food or non-food industries. Modifications of the fatty acid compositions have been sought after for at least a century in order to provide optimal oil products for human nutrition and chemical (e.g., oleochemical) uses (Gunstone, 1998, Prog Lipid Res 37:277; Broun et al., 1999, Annu Rev Nutr 19:107; Jaworski et al, 2003, Curr Opin Plant Biol 6:178). Low levels of saturated fatty acids are beneficial for health. High oleic low linolenic canola oil improves frying performance.

Optimizing the oil characteristics in Brassica oilseeds has been achieved by breeding as well as biotechnological approaches.

### SUMMARY OF THE PREFERRED EMBODIMENTS OF THE INVENTION

In a first embodiment of the invention, a method is provided for enhancing oil characteristics in a Brassica oilseed plant, said method comprising growing Brassica oilseed plants, and harvesting the seeds by straight cutting. In a further embodiment, said Brassica oilseed plants are podshatter resistant, such as Brassica oilseed plants containing a modified Indehiscent gene.

In another embodiment, the podshattering is inhibited by application of pod sealants to the growing Brassica oilseed plants.

Another embodiment provides a method to increase oil quantity in a Brassica oilseed plant, said method comprising growing Brassica oilseed plants, such as Brassica oilseed plants being podshatter resistant, and harvesting the seeds by straight cutting, whereas another embodiment provides a method to reduce the levels of saturated fatty acids in the oil of a Brassica oilseed plant, or for increase the oil healthiness, said method comprising growing Brassica oilseed plants, such as Brassica oilseed plants being podshatter resistant, and harvesting the seeds by straight cutting.

In yet another embodiment, said Brassica oilseed plant is Brassica napus, such as a hybrid Brassica napus plant. In another aspect, said Brassica oilseed plant is resistant to a herbicide.

In another embodiment, the method according to the invention further comprises treating the growing Brassica oilseed plants with a herbicide.

In another embodiment, a method is provided of producing Brassica oilseed oil with enhanced characteristics, said method comprising growing Brassica oilseed plants, harvesting the seeds by straight cutting, and extracting the oil from said seeds. In another embodiment, the enhanced characteristic is improved health.

A further embodiment provides the use of the seed obtained using the methods according to the invention for the production of oil with enhanced characteristics, and the use of the oil obtained using the methods according to the invention as food ingredient.

### DETAILED DESCRIPTION

The current invention is based on the observation that seeds of podshatter resistant Brassica oilseed plants that are harvested using straight cutting have improved oil characteristics as compared to plants harvested using swathing.

In a first embodiment of the invention, a method is provided for enhancing oil characteristics in a Brassica oilseed plant, said method comprising growing Brassica oilseed plants, and harvesting the seeds by straight cutting.

Brassica oilseed plants, also called rapeseed, as used herein are Brassica plants which can be cultivated for the seed oil. Brassica oilseeds encompass Brassica napus, Brassica juncea, Brassica carinata and some types of Brassica rapa.

Brassica oilseed plants can be canola plants. To use the name canola, an oilseed plant must meet the following internationally regulated standard: “Seeds of the genus Brassica (Brassica napus, Brassica rapa or Brassica juncea) from which the oil shall contain less than 2% erucic acid in its fatty acid profile and the solid component shall contain less than 30 micromoles of any one or any mixture of 3-butenyl glucosinolate, 4-pentenyl glucosinolate, 2-hydroxy-3 butenyl glucosinolate, and 2-hydroxy-4-pentenyl glucosinolate per gram of air-dry, oil-free solid.”

Straight cutting is a harvesting process in which the plants are left standing until harvest, in a simultaneous approach of cutting the plants and threshing the seeds from the seed pods on the plant. Straight cutting can be performed by direct combine harvesting. As opposed to straight cutting, swathing is a process in which the plants are cut first, and left on the field to dry before being harvested.

Straight cutting allows the plants to further mature as compared to swathing. However, a disadvantage of straight cutting is that the pods may open during ripening, resulting in yield loss due to podshattering. Straight cutting is usually later than BBCH stage 97 when the seed is drying such as when the moisture content of the seed is 10.5% or lower; or when the seed is fully cured.

Suitable to the invention is straight cutting at BBCH stage 97 or later. Also suitable is straight cutting when the moisture content of the seed is 10.5% or lower. Also suitable is straight cutting when the seed is fully cured.

Swathing can take place at BBCS stage 86-87, or at between 30% and 70% seed color change, or between 60%- 70% seed color change, or up to an average of 70% seed color change, or or up to an average of 60% seed color change.

After swathing, the plants can be left on the field for about 8-21 days, or for about 10-14 days, or for 10-14 days, or until the seed is fully cured.

Therefore, in a preferred embodiment, said Brassica oilseed plants are podshatter resistant, such as Brassica oilseed plants containing a modified Indehiscent gene.

Podshatter resistant Brassica oilseed plants can be obtained in many ways. On the one hand, podshatter resistant Brassica oilseed plants can be plants that are naturally less prone to podshattering. For example, Brassica juncea, Brassica carinata and Brassica rapa are less prone to pod shattering as compared to Brassica napus.

Podshatter resistant plants can also be obtained via breeding for increased podshatter resistance. The genetic basis of the increased podshatter resistance may, or may not, be known. Knowledge on genetic information and QTLs associated with regulation of podshatter can be employed to breed for podshatter resistant Brassica oilseed varieties. Examples of QTLs regulating variation for podshattering are described in Rahman et al (2014), PLOS ONE 9: e101673; Rahman et al (2017) Front Plant Sci 8:1765; Sra et al (2019) Mol Biol Rep 46:1227; or Kaur et al (2020) Mol Biol Rep 47:2963. Podshatter resistant canola and QTLs associated with podshatter resistance have been described in WO2016011146.

Podshatter resistant Brassica oilseed plants can be plants in which a gene affecting podshatter resistance has been modified. Such plants can, for example, be plants with a heterologous gene affecting podshatter resistance, such as podshatter resistance associated with the Ogura restorer of fertility (WO 2017/025420) or modification of biological pathways affecting podshattering (WO 2011/157976). Such plants can also be plants in which expression of endogenous genes is modified (such as described in WO 2004/113542 or WO 1996/030529 or WO 2011/157976). Podshatter resistant Brassica oilseed plants can also be plants with modified endogenes, such as Alcatraz genez (WO 2012/084742), Indehiscent genes (WO 2006/009649, WO 2009/068313, or WO 2010/006732), or Shatterproof genes (2019/140009).

Podshatter resistant Brassica oilseed plants may contain a c to t substitution at position 364 of SEQ ID NO: 1; a g to a substitution at position 307 of SEQ ID NO: 1 combined with a g to a substitution at position 380 of SEQ ID NO: 1; a c to t substitution at position 148 of SEQ ID NO: 3, or a c to t substitution at position 403 of SEQ ID NO: 3 (the ind-a1-EMS01, the ind-a1-EMS05 mutation, the ind-c1-EMS01 mutation or the ind-c1-EMS03 mutation, respectively of WO2009/068313), or a Val to Met substitution at position 124 of SEQ ID NO: 2, or a Gly to Ser substitution at position 146 of SEQ ID NO: 2, or an Ala to Val substitution at position 159 of SEQ ID NO: 2, or a Thr to Met substitution at position 136 of SEQ ID NO: 4, or an Ala to Thr substitution at position 139 of SEQ ID NO: 4, or an Arg to Cys substitution at position 142 of SEQ ID NO: 4 (the ind-a1-EMS06, the ind-a1-EMS09 mutation, the ind-a1-EMS13 mutation, the ind-c1-EMS08 mutation, the ind-c1-EMS09 mutation or the ind-c1-EMS04 mutation, respectively of WO 2010/006732.

Such endogenous genes may be modified using genome editing strategies, or mutagenesis techniques.

Genome editing, also called gene editing, genome engineering, as used herein, refers to the targeted modification of genomic DNA in which the DNA may be inserted, deleted, modified or replaced in the genome. Genome editing may use sequence-specific enzymes (such as endonuclease, nickases, base conversion enzymes) and/or donor nucleic acids (e.g. dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2c1, C2c3, certain Argonaut-based systems (see e.g. Osakabe and Osakabe, Plant Cell Physiol. 2015 Mar;56(3):389-400; Ma et al., Mol Plant. 2016 Jul 6;9(7):961-74; Bortesie et al., Plant Biotech J, 2016, 14; Murovec et al., Plant Biotechnol J. 15:917-926, 2017; Nakade et al., Bioengineered Vol 8, No.3:265-273, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease. Donor nucleic acids can also be used as such for genome editing without DNA break induction to introduce a desired change into the genomic DNA.

Mutagenesis, as used herein, refers to the process in which plant cells (e.g., a plurality of Brassica seeds or other parts, such as pollen, etc.) are subjected to a technique which induces mutations in the DNA of the cells, such as contact with a mutagenic agent, such as a chemical substance (such as ethylmethylsulfonate (EMS), ethylnitrosourea (ENU), etc.) or ionizing radiation (neutrons (such as in fast neutron mutagenesis, etc.), alpha rays, gamma rays (such as that supplied by a Cobalt 60 source), X-rays, UV-radiation, etc.), or a combination of two or more of these. Thus, the desired mutagenesis may be accomplished by use of chemical means such as by contact of one or more plant tissues with ethylmethylsulfonate (EMS), ethylnitrosourea, etc., by the use of physical means such as x-ray, etc, or by gamma radiation, such as that supplied by a Cobalt 60 source. While mutations created by irradiation are often large deletions or other gross lesions such as translocations or complex rearrangements, mutations created by chemical mutagens are often more discrete lesions such as point mutations. For example, EMS alkylates guanine bases, which results in base mispairing: an alkylated guanine will pair with a thymine base, resulting primarily in G/C to A/T transitions. Following mutagenesis, Brassica plants are regenerated from the treated cells using known techniques. For instance, the resulting Brassica seeds may be planted in accordance with conventional growing procedures and following self-pollination seed is formed on the plants. Alternatively, doubled haploid plantlets may be extracted to immediately form homozygous plants, for example as described by Coventry et al. (1988, Manual for Microspore Culture Technique for Brassica napus. Dep. Crop Sci. Techn. Bull. OAC Publication 0489. Univ. of Guelph, Guelph, Ontario, Canada). Additional seed that is formed as a result of such self-pollination in the present or a subsequent generation may be harvested and screened for the presence of mutant alleles. Several techniques are known to screen for specific mutant alleles, e.g., Deleteagene™ (Delete-a-gene; Li et al., 2001, Plant J 27: 235-242) uses polymerase chain reaction (PCR) assays to screen for deletion mutants generated by fast neutron mutagenesis, TILLING (targeted induced local lesions in genomes; McCallum et al., 2000, Nat Biotechnol 18:455-457) identifies EMS-induced point mutations, etc.

Podshatter resistant Brassica oilseed plants as used herein refers to plants having podshatter resistance.

The term “Podshatter Resistance” means the resistance to silique shattering is observed at seed maturity. Podshatter Resistance can be measured in the field at maturity and assessed on a scale of 1-5, where 1=no shatter loss and 5=significant shatter loss.

Podshatter resistant plants may have a podshatter resistance value of 3 or lower, or of 2 or lower, or between 1 and 2, or between 1 and 1.8, or between 1 and 1.5, or between 1 and 1.4, or between 1.1 and 1.4.

Podshatter resistance can also be measured by inspection of the pods with naked eye, or with a Manual Impact Test, or with a Random Impact Test as described, for example, in WO2010/006732. In a random Impact Test (RIT) 20 intact mature pods can be placed together with six steel balls of 12.5 mm diameter in a cylindrical container of diameter 20 cm with its axis vertical. The container is then subjected to simple harmonic motion of frequency 4.98 Hz and of stroke 51 mm in the horizontal plane. The pods, checked for soundness before the test, are shaken for cumulative times of 10, 20, 40, and, if more than 50% of pods remained intact, 80 s. The drum is opened after each period and the number of closed pods counted. The pods are examined and classed as “closed” if the dehiscence zone of both valves is still closed. Thus the pods are classed as “opened” if one or both of the valves are detached, so that the seed had been released. If the majority of the pods is broken or damaged without opening of the dehiscence zone, the sample is marked “uncountable”. To give each point equal weighing, the data are made evenly spaced in the independent variable, time, by adding 1 and taking log 10. The percentage of pods opened p is transformed by the logit transformation, i.e. logit p=log e(p/100−p). A linear model is then fitted to the transformed time and percentage data and used to estimate the pod sample half-life (LD50).

Podshatter resistant plants grown in the glasshouse may have a pod sample half life a RIT of more than 10 seconds, or of more than 15 seconds, or of more than 20 seconds, or of more than 30 seconds, or of more than 40 seconds, or between 10 and 70 seconds, between 15 and 70 seconds, between 10 and 60 seconds, between 10 and 50 seconds, between 20 and 60 seconds, between 20 and 50 seconds, between 40 and 60 seconds, of about 57 seconds.

Podshatter resistant plants can be podshatter resistant oilseed or canola varieties, such as InVigor L345PC (BASF), InVigor L233P (BASF), InVigor L234PC (BASF), InVigor L255PC (BASF), InVigor R 4022P (BASF), InVigor R 5520P (BASF) 74-44 RR (Dekalb), 75-65 RR (Dekalb) 75-65 RR (Dekalb); DKLL 82 SC (Dekalb); DKTF 92 SC (Dekalb); DKTF 96 SC (Dekalb); DKTF 97 CRSC (Dekalb); DKTF 99 SC (Dekalb); DKTFLL 21 SC (Dekalb); CS2600 CR-T (Canterra Seeds); CS2400 (Canterra Seeds); 6090 RR (Brett Young); 2024 CL (Brevant); B2030MN (Brevant); B3010M (Brevant); D3158CM (Brevant); 45CM36 (Pioneer Hi-Bred); 45CM39 (Pioneer Hi-Bred); 45H42 (Pioneer Hi-Bred); 45M35 (Pioneer Hi-Bred); 45M38 (Pioneer Hi-Bred); P505MSL (Pioneer Hi-Bred); P506ML (Pioneer Hi-Bred); PV 560 GM (Proven Seeds); PV660 LCM (Proven Seeds); PV 761 TM (Proven Seeds).

In another embodiment, the podshattering is inhibited by application of pod sealants to the growing Brassica oilseed plants.

Pod sealants compounds, such as polymer sprays, that prevent the pods from splitting open during ripening. An example of a pod sealant is Pod Ceal DC® (Miller Chemical) or Pod-Stik® (Loveland products).

Another embodiment provides a method to increase oil quantity in a Brassica oilseed plant, said method comprising growing Brassica oilseed plants, such as Brassica oilseed plants being podshatter resistant, and harvesting the seeds by straight cutting, whereas another embodiment provides a method to reduce the levels of saturated fatty acids in the oil of a Brassica oilseed plant, or for increase the oil healthiness, said method comprising growing Brassica oilseed plants, such as Brassica oilseed plants being podshatter resistant, and harvesting the seeds by straight cutting.

Enhancing oil characteristics as used herein refers to modification of oil characteristics in a beneficial manner. This can be beneficial with regard to yield, such as increased oil content, or with regard to beneficial oil quality parameters for improved health, or improved chemical properties such as stability or viscosity. Beneficial oil quality characteristics can be, for example, decreased levels of glucosinolates, increased levels of oleic acid (C18:1), reduced levels of linolenic and linoleic acid (C18:3 and C18:2, respectively), or reduced levels of saturated fatty acids.

An increase in oil quantity can be an increase in oil quantity with at least 0.5%, or at least 1%, or about 1.3%, or 1.3%.

Oil quantity can be measured using Near Infrared Spectroscopy (NIR) as known in the art.

The reduction in levels of saturated fatty acids can be a reduction with at least 0.5%, or at least 1%, or at least 1.5%, or at least 2%, or at least 2.5%, or at least 2.7%, or about 3%.

Levels of saturated fatty acids can be measured using Capillary Gas-Liquid Chromatography (CG-LC) as known in the art and or as described in WO2009/007091.

Saturated fatty acids, as used herein, are fatty acids which do not have C═C double bonds. They have the same formula CH3(CH2)nCOOH, with variations in “n”. Examples of saturated fatty acids are: Caprylic acid (CH3(CH2)6COOH/C8:0); Capric acid (CH3(CH2)8COOH; C10:0); Lauric acid (CH3(CH2)10COOH; C12:0); Myristic acid (CH3(CH2)12COOH; C14:0); Palmitic acid (CH3(CH2)14COOH; C16:0); Stearic acid (CH3(CH2)16COOH; C18:0); Arachidic acid (CH3(CH2)18COOH; C20:0); Behenic acid (CH3(CH2)20COOH; C22:0); Lignoceric acid (CH3(CH2)22COOH; C24:0); and Cerotic acid (CH3(CH2)24COOH; C26:0).

The reduction in levels of saturated fatty acids can be a reduction in the levels of C12:0, and/or C14:0, and/or C16:0, and/or C18:0, and/or C20:0, and/or C22:0, and/or C24:0, such as a reduction in the levels of C16:0, and/or C18:0, and/or C20:0, and/or C22:0, and/or C24:0.

The enhanced oil characteristics, such as the increased oil content, or the reduced levels of saturated fatty acid, are as compared to the same plants grown under the same conditions harvested by swathing. Swathing may have occurred about 10-14 days earlier than straight harvesting.

In yet another embodiment, said Brassica oilseed plant is Brassica napus, such as a hybrid Brassica napus plant. In another aspect, said Brassica oilseed plant is resistant to a herbicide.

In another embodiment, the method according to the invention further comprises treating the growing Brassica oilseed plants with a herbicide.

A “hybrid plant” is a plant which is typically created in a cross between two inbred parent lines. A hybrid plant has a high level of heterozygosity. A hybrid plant may or may not show hybrid vigor (or heterosis), i.e. an increase in characteristics, such as yield, over those of its parents.

Hybrid seed is the seed resulting from a pollination of an inbred female plant with pollen from an inbred male plant. When planted, hybrid seed grows into a hybrid plant.

In order to produce pure hybrid seeds one of the parental lines is male sterile and is pollinated with pollen of the other line. By growing parental lines in rows and only harvesting the F1 seed of the male sterile parent, pure hybrid seeds are produced. To generate male sterile parental lines, the system as described in EP 0,344,029 or U.S. Pat. No. 6,509,516 may be used, wherein a gene encoding a phytotoxic protein (barnase) is expressed under the control of a tapetum specific promoter, such as TA29, ensuring selective destruction of tapetum cells. Transformation of plants with the chimeric gene pTA29:barnase results in plants in which pollen formation is completely prevented [Mariani et al. (1990), Nature 347: 737-741]. Cytochemical and histochemical analysis of another development of Brassica napus plants comprising the chimeric pTA29-barnase gene is described by De Block and De Brouwer [(1993), Planta 189:218-225]. To restore fertility in the progeny of a male-sterile plant the male-sterile plant (MS parent) is crossed with a transgenic plant (RF parent) carrying a fertility-restorer gene, which when expressed is capable of inhibiting or preventing the activity of the male-sterility gene [U.S. Pat. Nos. 5,689,041; 5,792,929; De Block and De Brouwer, supra]. The use of co-regulating genes in the production of male-sterile plants to increase the frequency of transformants having good agronomical performance is described in WO96/26283. Typically, when the sterility DNA encodes a barnase, the co-regulating DNA will encode a barstar, preferably an optimized barstar gene is used as described in published PCT patent application WO 98/10081. It is understood that different promoters may be used to drive barnase expression in order to render the plant male sterile. Likewise, barstar may be operably linked to different promoters, such as 35S from Cauliflower mosaic virus.

Male sterile plants can also be generated using other techniques, such as cytoplasmic male sterility/restorer systems [e.g. the Ogura system, published US patent application 20020032916, U.S. Pat. No. 6,229,072, WO97/02737, U.S. Pat. No. 5,789,566 or the Polima system of U.S. Pat. No. 6,365,798, WO98/54340 or the Kosena system of WO95/09910, U.S. Pat. No. 5,644,066].

The Brassica oilseeds plant can be a winter oilseed rape or spring oilseed rape.

“Winter oilseed rape” or “WOSR” is Brassica oilseed which is planted in late summer to early autumn, overwinters, and is harvested the following summer. WOSR generally requires vernalization to flower.

“Spring oilseed rape” or “SOSR” is Brassica oilseed which is planted in the early spring and harvested in late summer. SOSR does not require vernalization to flower.

The Brassica oilseed plant resistant to a herbicide may comprise a gene conferring herbicide resistance. Said gene comferring herbicide resistance may be the bar or pat gene, which confer resistance to glufosinate ammonium (Liberty®, Basta® or Ignite®) [EP 0 242 236 and EP 0 242 246 incorporated by reference]; or any modified EPSPS gene, such as the 2mEPSPS gene from maize [EPO 508 909 and EP 0 507 698 incorporated by reference], or glyphosate acetyltransferase, or glyphosate oxidoreductase, which confer resistance to glyphosate (RoundupReady®), or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola.

Further, the Brassica oilseed plants may additionally contain an endogenous or a transgene which confers increased oil content or improved oil composition, such as a 12:0 ACP thioesteraseincrease to obtain high laureate, which confers pollination control, such as such as barnase under control of an anther-specific promoter to obtain male sterility, or barstar under control of an anther-specific promoter to confer restoration of male sterility, or such as the Ogura cytoplasmic male sterility and nuclear restorer of fertility.

The Brassica oilseed plants which additionally contain a gene which confers resistance to glufosinate ammonium (Liberty®, Basta® or Ignite®) may contain a gene coding for a phosphinothricin-N-acetyltransferase (PAT) enzyme, such as a coding sequence of the bialaphos resistance gene (bar) of Streptomyces hygroscopicus. Such plants may, for example, comprise the elite events MS-BN1 and/or RF-BN1 as described in WO01/41558, or elite event MS-B2 and/or RF-BN1 as described in WO01/31042 or in WO2014/170387, or any combination of these events.

The Brassica oilseed plants which contain a gene which confers resistance to glyphosate (RoundupReady®) may contain a glyphosate resistant EPSPS, such as a CP4 EPSPS, or an N-acetyltransferase (gat) gene. Such plants may, for example, comprise the elite event RT73 as described in WO02/36831, or elite event MON88302 as described in WO11/153186, or event DP-073496-4 as described in WO2012/071040.

Said Brassica oilseed plants which contain a gene which confers resistance to glufosinate ammonium can be treated with glufosinate or glufosinate ammonium herbicide. Said Brassica oilseed plants which contain a gene which confers resistance to glyphosate can be treated with glyphosate herbicide, and the herbicide is glufosinate or glufosinate ammonium or glyphosate. Said Brassica oilseed plants which contain a gene which confers resistance to imidazolinones can be treated with imazamox or imidazolinone herbicide.

The methods as described herein for enhancing oil characteristics can also be applied in methods to produce Brassica oilseed oil with enhanced characteristics, said methods comprising the steps of the methods as described herein for enhancing oil characteristics, further comprising the step of extracting the oil from said harvested seeds.

In another embodiment, a method is provided of producing Brassica oilseed oil with enhanced characteristics, said method comprising growing Brassica oilseed plants, harvesting the seeds by straight cutting, and extracting the oil from said seeds. In another embodiment, the enhanced characteristic is improved health.

Improved health can be reduced levels of blood cholesterol.

A further embodiment provides the use of the seed obtained using the methods according to the invention for the production of oil with enhanced characteristics, and the use of the oil obtained using the methods according to the invention as food ingredient.

All patents, patent applications, and publications or public disclosures (including publications on internet) referred to or cited herein are incorporated by reference in their entirety.

The sequence listing contained in the file named “200002_Std26.xml”, which is 9 kilobytes (size as measured in Microsoft Windows®), contains 4 sequences SEQ ID NO: 1 through SEQ ID NO: 4 is filed herewith by electronic submission and is incorporated by reference herein.

In the description and examples, reference is made to the following sequences:
SEQ ID No. 1: B. napus IND-A1 coding sequence
SEQ ID No. 2: B. napus IND-A1 protein sequence
SEQ ID No. 3: B. napus IND-C1 coding sequence
SEQ ID No. 4: B. napus IND-C1 protein sequence

### EXAMPLES

### 1. Obtaining Podshatter Resistant B. Napus Lines

Podshatter resistant B. napus lines were obtained as described in WO2010/006732. The podshatter resistant mutant ind-c1-EMS09 of WO2010/006732 was used for further analysis in this study. Ind-c1-EMS09 contains a g to a substitution at position at position 415 of the IND-C1 coding sequence (SEQ ID NO: 3), which results in a Ala to Thr substitution at position 139 of the encoded IND-C1 protein (SEQ ID NO: 4). B. napus plants comprising the ind-c1-EMS09 contain an increased podshatter resistance, as shown by the force to open the pods, as well as an increased yield (WO2010/006732).

The ind-c1-EMS09 was introgressed into several oilseed rape varieties, including elite spring oilseed rape varieties. Two elite hybrid spring oilseed rape varieties (variety 130 and variety 122) containing ind-c1-EMS09 were used for further analysis.

Variety 130 is an early maturing variety which is suitable for the growing zones in Western Canada, whereas Variety 122 is late maturing suitable for mid-to long growing zones in Western Canada.

### 2. Analysis of Podshatterring Properties of the Podshatter Resistant Varieties in the Field

The podshatter resistance of varieties 130 and 122 were tested in the field. The plants were grown to maturity and the podshatter resistance was scored on a scale 1 to 5, wherein 1=all pods intact at harvest time, and 5=a significant amount of pods on the ground.

As an average over 4 different locations, variety 122 had a shatter resistance value of 1.4 as compared to 2.1 for a non shatter resistant check. Variety 130 had a shatter resistance value of 1.14 as compared to 1.86 for a non shatter resistant check.

### 3. Analysis of Seed Properties of Podshatter Resistant Varieties in the Field

The two podshatter resistant varieties 130 and 122 were grown in the field during three growing seasons at up to 12 different locations in 65 plots in total. 13 plots were omitted from the analysis because of poor data quality due to adverse conditions or suboptimal plot setup. The different locations were in three growing zones: short season zone (SSZ), mid season zone (MSZ) and long season zone (LSZ). Two different harvesting methods were used in each field trial: straight cutting and swathing. Swathing took place at BBCH stage 86-87 and the plants were left 10-14 days on the field before harvesting. Straight cutting took place around BBCH stage 97.

The harvested grain was analyzed for the following parameters:
Grain yield (KG/HA)
Oil content (percentage)
Protein content (percentage)
Days to maturity
Fatty acid composition (C12:0, C14:09, C16:0, C16:1, C18:0, C18:1, C18:2, C18:3, C20:0, C20:1, C20:2, C22:0, C22:1, C24:0. C24:1; (% of oil weight in seed) (analyzed for 26 of the 52 plots)
Glucosinolate content (μmole/gram seed)

Oil content, protein content and glucosinolate content were determined using Near-Infrared Spectroscopy.

Days to maturity was determined based on the criteria that seed colour change on the main raceme was 60%, and several pods per plot (from middle of the way up the main raceme to 2/3 of the way to the top) should be opened.

The fatty acid composition of the seed oil was determined by extracting the fatty acyls from the seeds and analyzing their relative levels in the seed oil by capillary gas-liquid chromatography as described in WO09/007091. Seed quality parameters were obtained through GC analysis.

### 4. Seed Properties of Podshatter Resistant Varieties After Straight Cutting and Swathing in the Field

The properties of the harvested seed after straight cutting and swathing are shown in Tables 1 and 2.

TABLE 1a
seed properties of variety 130 harvested by straight cutting (SC) or swathing (SW). SSZ: Short season zone; MSZ: Mid season zone;
LSZ: Long season zone; DMAT: days to maturity. Av: average across locations; Δ: difference of straight cutting versus swathing.

| | | | | | | Relative | | Oil | | Protein | | | Gluc |
|---|---|---|---|---|---|---|---|---|---|---|---|---|---|
| | | | | | Yield | Yield | | Difference | | Difference | | GLUCOSIN- | Difference |
| Year | Zone | Location | variety | Treatment | KG/HA | Difference | OIL | (Units) | PROTEIN | (Units) | DMAT | OLATES | (Units) |
| 2017 | LSZ | 1 | 130 | SC | 5.33 | 1.15 | 46.70 | 0.77 | 47.50 | 0.35 | 90.00 | 10.96 | 2.35 |
| 2017 | LSZ | 1 | 130 | SW | 4.64 | | 45.93 | | 47.15 | | 89.00 | 8.61 | |
| 2017 | MSZ | 2 | 130 | SC | 5.76 | 1.18 | 45.65 | 0.25 | 48.11 | 0.21 | 89.50 | 8.28 | −1.42 |
| 2017 | MSZ | 2 | 130 | SW | 4.88 | | 45.40 | | 47.90 | | 89.50 | 9.70 | |
| 2017 | SSZ | 3 | 130 | SC | 5.47 | 1.06 | 49.05 | 0.35 | 41.41 | 0.57 | 93.50 | 9.47 | −0.05 |
| 2017 | SSZ | 3 | 130 | SW | 5.17 | | 48.70 | | 40.84 | | 94.50 | 9.52 | |
| 2017 | SSZ | 4 | 130 | SC | 5.26 | 1.17 | 46.21 | 0.14 | 50.12 | 1.28 | 102.50 | 9.53 | 2.31 |
| 2017 | SSZ | 4 | 130 | SW | 4.49 | | 46.07 | | 48.84 | | 103.00 | 7.22 | |
| 2017 | MSZ | 5 | 130 | SC | 6.00 | 1.03 | 47.50 | 0.50 | 46.38 | −0.12 | 93.50 | 7.37 | −0.09 |
| 2017 | MSZ | 5 | 130 | SW | 5.84 | | 47.00 | | 46.50 | | 94.00 | 7.46 | |
| 2017 | LSZ | 1 | 130 | SC | 4.66 | 1.19 | 46.99 | 1.58 | 46.09 | −1.18 | 87.00 | 7.37 | −3.58 |
| 2017 | LSZ | 1 | 130 | SW | 3.90 | | 45.41 | | 47.27 | | 87.00 | 10.95 | |
| 2017 | MSZ | 2 | 130 | SC | 4.73 | 1.20 | 46.70 | 0.15 | 46.43 | 0.80 | 90.00 | 7.82 | −1.65 |
| 2017 | MSZ | 2 | 130 | SW | 3.95 | | 46.55 | | 45.63 | | 89.50 | 9.47 | |
| 2017 | MSZ | 6 | 130 | SC | 5.17 | 1.14 | 46.85 | 0.85 | 47.32 | −0.09 | 93.50 | 8.88 | 0.23 |
| 2017 | MSZ | 6 | 130 | SW | 4.53 | | 46.00 | | 47.41 | | 94.50 | 8.65 | |
| 2017 | MSZ | 7 | 130 | SC | 5.47 | 0.98 | 47.00 | 0.30 | 46.70 | 0.46 | 95.00 | 8.83 | 0.00 |
| 2017 | MSZ | 7 | 130 | SW | 5.60 | | 46.70 | | 46.24 | | 96.00 | 8.83 | |
| 2017 | MSZ | 5 | 130 | SC | 5.70 | 1.00 | 47.60 | 0.95 | 46.18 | −1.05 | 93.00 | 7.59 | −0.55 |
| 2017 | MSZ | 5 | 130 | SW | 5.68 | | 46.65 | | 47.23 | | 94.00 | 8.14 | |
| 2017 | LSZ | 1 | 130 | SC | 4.39 | 0.99 | 46.54 | 0.62 | 47.16 | 0.37 | 87.00 | 8.77 | −2.56 |
| 2017 | LSZ | 1 | 130 | SW | 4.43 | | 45.92 | | 46.79 | | 87.50 | 11.33 | |
| 2017 | MSZ | 2 | 130 | SC | 5.58 | 1.27 | 46.25 | 0.80 | 47.16 | 1.52 | 90.50 | 8.97 | −2.88 |
| 2017 | MSZ | 2 | 130 | SW | 4.40 | | 45.45 | | 45.64 | | 90.00 | 11.85 | |
| 2017 | MSZ | 8 | 130 | SC | 6.03 | 1.02 | 47.97 | −0.60 | 48.32 | 1.92 | 99.00 | 9.70 | 0.89 |
| 2017 | MSZ | 8 | 130 | SW | 5.90 | | 48.57 | | 46.40 | | 99.50 | 8.81 | |
| 2017 | SSZ | 4 | 130 | SC | 5.66 | 1.59 | 46.39 | 0.30 | 49.56 | 1.67 | 102.50 | 9.69 | −0.43 |
| 2017 | SSZ | 4 | 130 | SW | 3.57 | | 46.09 | | 47.89 | | 105.50 | 10.12 | |
| 2018 | LSZ | 1 | 130 | SC | 4.65 | 1.34 | 43.85 | 0.72 | 51.05 | −0.35 | 84.50 | 12.54 | −2.11 |
| 2018 | LSZ | 1 | 130 | SW | 3.47 | | 43.13 | | 51.40 | | 82.50 | 14.65 | |
| 2018 | MSZ | 9 | 130 | SC | 4.86 | 1.17 | 45.25 | 1.45 | 47.20 | 0.49 | 85.00 | 12.90 | 3.34 |
| 2018 | MSZ | 9 | 130 | SW | 4.15 | | 43.80 | | 46.71 | | 86.00 | 9.56 | |
| 2018 | MSZ | 10 | 130 | SC | 3.07 | 1.04 | 44.18 | −0.84 | 52.47 | 3.56 | 91.50 | 12.44 | −0.32 |
| 2018 | MSZ | 10 | 130 | SW | 2.95 | | 45.02 | | 48.91 | | 95.00 | 12.76 | |
| 2018 | LSZ | 1 | 130 | SC | 3.78 | 0.89 | 42.88 | −0.80 | 51.12 | 0.48 | 83.50 | 13.82 | 1.17 |
| 2018 | LSZ | 1 | 130 | SW | 4.23 | | 43.68 | | 50.64 | | 85.00 | 12.65 | |
| 2018 | MSZ | 9 | 130 | SC | 4.72 | 1.12 | 43.50 | 0.55 | 47.61 | 0.54 | 84.50 | 6.63 | −0.46 |
| 2018 | MSZ | 9 | 130 | SW | 4.21 | | 42.95 | | 47.07 | | 84.00 | 7.09 | |
| 2018 | MSZ | 2 | 130 | SC | 5.70 | 1.08 | 46.30 | 0.05 | 49.62 | 0.32 | 86.50 | 11.35 | 0.92 |
| 2018 | MSZ | 2 | 130 | SW | 5.29 | | 46.25 | | 49.30 | | 87.50 | 10.43 | |
| 2018 | SSZ | 3 | 130 | SC | 4.48 | 1.07 | 46.45 | −0.50 | 42.11 | −0.78 | 100.50 | 6.68 | −1.05 |
| 2018 | SSZ | 3 | 130 | SW | 4.20 | | 46.95 | | 42.89 | | 101.00 | 7.73 | |
| 2018 | MSZ | 5 | 130 | SC | 2.81 | 1.03 | 45.35 | 0.03 | 51.23 | 4.41 | 84.00 | 11.85 | −0.84 |
| 2018 | MSZ | 5 | 130 | SW | 2.74 | | 45.32 | | 46.82 | | 85.50 | 12.69 | |
| 2018 | MSZ | 9 | 130 | SC | 4.41 | 0.98 | 45.15 | 0.70 | 45.38 | 0.23 | 84.50 | 9.15 | −1.10 |
| 2018 | MSZ | 9 | 130 | SW | 4.52 | | 44.45 | | 45.15 | | 86.00 | 10.25 | |
| 2018 | MSZ | 2 | 130 | SC | 6.05 | 1.04 | 45.70 | 0.10 | 50.36 | 1.01 | 89.50 | 11.85 | −0.23 |
| 2018 | MSZ | 2 | 130 | SW | 5.82 | | 45.60 | | 49.35 | | 90.00 | 12.08 | |
| 2018 | MSZ | 11 | 130 | SC | 3.63 | 1.29 | 41.75 | 1.69 | 52.81 | 2.61 | 92.50 | 12.67 | 0.75 |
| 2018 | MSZ | 11 | 130 | SW | 2.81 | | 40.06 | | 50.20 | | 92.50 | 11.92 | |
| 2019 | MSZ | 11 | 130 | SC | 4.37 | 1.29 | 48.25 | 3.75 | 44.88 | −0.52 | 113.50 | 9.95 | 0.95 |
| 2019 | MSZ | 11 | 130 | SW | 3.39 | | 44.50 | | 45.40 | | 113.50 | 9.00 | |
| 2019 | LSZ | 1 | 130 | SC | 4.01 | 1.49 | 46.15 | 2.05 | 48.66 | 3.31 | 82.50 | 8.65 | 0.00 |
| 2019 | LSZ | 1 | 130 | SW | 2.70 | | 44.10 | | 45.35 | | 81.50 | 8.65 | |
| 2019 | MSZ | 11 | 130 | SC | 4.56 | 1.12 | 48.05 | 1.21 | 45.03 | 0.83 | 110.00 | 10.71 | 0.73 |
| 2019 | MSZ | 11 | 130 | SW | 4.06 | | 46.84 | | 44.20 | | 108.50 | 9.98 | |
| 2019 | LSZ | 12 | 130 | SC | 2.74 | 1.05 | 46.20 | −0.05 | 45.26 | 1.08 | 89.00 | 8.01 | 0.78 |
| 2019 | LSZ | 12 | 130 | SW | 2.60 | | 46.25 | | 44.18 | | 86.50 | 7.23 | |
| 2019 | LSZ | 1 | 130 | SC | 4.18 | 0.98 | 45.20 | −0.60 | 47.45 | 1.55 | 84.00 | 8.14 | −0.87 |
| 2019 | LSZ | 1 | 130 | SW | 4.28 | | 45.80 | | 45.90 | | 85.00 | 9.01 | |
| 2019 | MSZ | 11 | 130 | SC | 4.37 | 1.12 | 47.75 | 2.04 | 44.47 | −0.42 | 108.00 | 9.86 | −0.67 |
| 2019 | MSZ | 11 | 130 | SW | 3.90 | | 45.71 | | 44.89 | | 109.00 | 10.53 | |
| | | | Av | SC | 4.76 | | 46.11 | | 47.59 | | 92.26 | 9.69 | |
| | | | Av | SW | 4.27 | | 45.51 | | 46.78 | | 92.66 | 9.90 | |
| | | | Δ | units | 0.49 | | 0.60 | | 0.81 | | −0.40 | −0.21 | |
| | | | Δ | % | 11.48 | | 1.32 | | 1.73 | | −0.43 | −2.12 | |

TABLE 1b
seed properties of variety 130 harvested by straight cutting (SC) or swathing (SW). SatFAT:
total saturated fatty acids. The fatty acid composition is given in % of oil weight in the
seed. Av: average across locations; Δ: difference of straight cutting versus swathing.

| Year | Zone | Location | variety | Treatment | SatFAT | C12_0 | C14_0 | C16_0 | C18_0 | C20_0 | C22_0 | C24_0 |
|---|---|---|---|---|---|---|---|---|---|---|---|---|
| 2017 | MSZ | 2 | 130 | SC | 6.54 | 0.01 | 0.05 | 3.59 | 1.80 | 0.61 | 0.29 | 0.20 |
| 2017 | MSZ | 0 | 130 | SW | 6.87 | 0.01 | 0.05 | 3.73 | 1.88 | 0.64 | 0.33 | 0.24 |
| 2017 | SSZ | 3 | 130 | SC | 6.70 | 0.01 | 0.05 | 3.74 | 1.83 | 0.60 | 0.31 | 0.17 |
| 2017 | SSZ | 3 | 130 | SW | 7.13 | 0.01 | 0.05 | 3.95 | 1.92 | 0.64 | 0.34 | 0.24 |
| 2017 | MSZ | 5 | 130 | SC | 6.69 | 0.01 | 0.05 | 3.69 | 1.84 | 0.62 | 0.33 | 0.17 |
| 2017 | MSZ | 5 | 130 | SW | 6.78 | 0.01 | 0.05 | 3.75 | 1.85 | 0.63 | 0.33 | 0.17 |
| 2017 | MSZ | 2 | 130 | SC | 6.64 | 0.01 | 0.05 | 3.67 | 1.83 | 0.60 | 0.30 | 0.19 |
| 2017 | MSZ | 2 | 130 | SW | 6.86 | 0.01 | 0.06 | 3.80 | 1.86 | 0.63 | 0.31 | 0.21 |
| 2017 | MSZ | 6 | 130 | SC | 6.62 | 0.01 | 0.04 | 3.58 | 1.91 | 0.61 | 0.29 | 0.18 |
| 2017 | MSZ | 6 | 130 | SW | 6.84 | 0.01 | 0.05 | 3.66 | 1.95 | 0.65 | 0.32 | 0.21 |
| 2017 | MSZ | 7 | 130 | SC | 6.62 | 0.01 | 0.05 | 3.65 | 1.83 | 0.60 | 0.30 | 0.19 |
| 2017 | MSZ | 7 | 130 | SW | 6.88 | 0.01 | 0.05 | 3.77 | 1.90 | 0.63 | 0.32 | 0.21 |
| 2017 | MSZ | 5 | 130 | SC | 6.67 | 0.01 | 0.05 | 3.68 | 1.84 | 0.61 | 0.32 | 0.17 |
| 2017 | MSZ | 5 | 130 | SW | 6.80 | 0.01 | 0.05 | 3.73 | 1.86 | 0.64 | 0.33 | 0.19 |
| 2017 | MSZ | 2 | 130 | SC | 6.53 | 0.01 | 0.05 | 3.59 | 1.80 | 0.59 | 0.30 | 0.20 |
| 2017 | MSZ | 2 | 130 | SW | 6.92 | 0.01 | 0.05 | 3.80 | 1.87 | 0.64 | 0.33 | 0.24 |
| 2018 | MSZ | 9 | 130 | SC | 7.05 | 0.01 | 0.05 | 3.94 | 1.92 | 0.62 | 0.30 | 0.23 |
| 2018 | MSZ | 9 | 130 | SW | 7.28 | 0.01 | 0.06 | 4.04 | 1.96 | 0.66 | 0.32 | 0.26 |
| 2018 | MSZ | 9 | 130 | SC | 7.02 | 0.01 | 0.06 | 3.95 | 1.88 | 0.62 | 0.29 | 0.22 |
| 2018 | MSZ | 9 | 130 | SW | 7.43 | 0.01 | 0.06 | 4.11 | 2.00 | 0.68 | 0.33 | 0.26 |
| 2018 | MSZ | 2 | 130 | SC | 6.83 | 0.01 | 0.05 | 3.75 | 1.91 | 0.61 | 0.30 | 0.23 |
| 2018 | MSZ | 2 | 130 | SW | 6.98 | 0.01 | 0.05 | 3.82 | 1.92 | 0.62 | 0.32 | 0.26 |
| 2018 | SSZ | 3 | 130 | SC | 6.85 | 0.01 | 0.04 | 4.15 | 1.52 | 0.58 | 0.36 | 0.20 |
| 2018 | SSZ | 3 | 130 | SW | 6.62 | 0.00 | 0.04 | 4.05 | 1.46 | 0.56 | 0.35 | 0.17 |
| 2018 | MSZ | 5 | 130 | SC | 6.75 | 0.01 | 0.05 | 3.63 | 1.97 | 0.59 | 0.28 | 0.24 |
| 2018 | MSZ | 5 | 130 | SW | 7.06 | 0.01 | 0.06 | 3.77 | 2.05 | 0.62 | 0.30 | 0.26 |
| 2018 | MSZ | 9 | 130 | SC | 7.20 | 0.01 | 0.06 | 3.91 | 2.02 | 0.66 | 0.31 | 0.25 |
| 2018 | MSZ | 9 | 130 | SW | 7.31 | 0.01 | 0.06 | 4.01 | 2.04 | 0.66 | 0.31 | 0.25 |
| 2018 | MSZ | 2 | 130 | SC | 6.63 | 0.00 | 0.04 | 3.64 | 1.86 | 0.60 | 0.30 | 0.21 |
| 2018 | MSZ | 2 | 130 | SW | 6.88 | 0.00 | 0.04 | 3.73 | 1.95 | 0.64 | 0.32 | 0.22 |
| | | | Av | SC | 6.76 | 0.01 | 0.05 | 3.74 | 1.85 | 0.61 | 0.31 | 0.20 |
| | | | Av | SW | 6.98 | 0.01 | 0.05 | 3.85 | 1.90 | 0.64 | 0.32 | 0.23 |
| | | | Δ | units | −0.22 | 0.00 | 0.00 | −0.11 | −0.05 | −0.03 | −0.01 | −0.03 |
| | | | Δ | % | −3.15 | 0.00 | 0.00 | −2.86 | −2.63 | −4.69 | −3.13 | −13.04 |

TABLE 1c
seed properties of variety 130 harvested by straight cutting (SC) or swathing (SW). The fatty acid composition is given
in % of oil weight in the seed. Av: average across locations; Δ: difference of straight cutting versus swathing.

| | | | | | | | | | | | C22_1 | |
|---|---|---|---|---|---|---|---|---|---|---|---|---|
| Year | Zone | Location | variety | Treatment | C16_1 | C18_1 | C18_2 | C18_3 | C20_1 | C20_2 | eruc | C24_1 |
| 2017 | MSZ | 2 | 130 | SC | 0.25 | 64.96 | 17.71 | 8.67 | 1.28 | 0.08 | 0.00 | 0.14 |
| 2017 | MSZ | 2 | 130 | SW | 0.28 | 63.92 | 18.42 | 8.62 | 1.27 | 0.07 | 0.00 | 0.17 |
| 2017 | SSZ | 3 | 130 | SC | 0.29 | 64.56 | 17.79 | 8.87 | 1.22 | 0.07 | 0.00 | 0.17 |
| 2017 | SSZ | 3 | 130 | SW | 0.33 | 63.30 | 18.45 | 8.87 | 1.21 | 0.07 | 0.00 | 0.18 |
| 2017 | MSZ | 5 | 130 | SC | 0.29 | 64.70 | 17.79 | 8.56 | 1.31 | 0.07 | 0.01 | 0.16 |
| 2017 | MSZ | 5 | 130 | SW | 0.30 | 63.66 | 18.22 | 9.03 | 1.30 | 0.07 | 0.02 | 0.16 |
| 2017 | MSZ | 2 | 130 | SC | 0.27 | 64.64 | 17.91 | 8.61 | 1.28 | 0.07 | 0.02 | 0.14 |
| 2017 | MSZ | 2 | 130 | SW | 0.29 | 63.98 | 18.18 | 8.68 | 1.26 | 0.07 | 0.02 | 0.16 |
| 2017 | MSZ | 6 | 130 | SC | 0.28 | 66.05 | 17.16 | 8.00 | 1.29 | 0.08 | 0.01 | 0.13 |
| 2017 | MSZ | 6 | 130 | SW | 0.29 | 65.64 | 17.22 | 8.05 | 1.31 | 0.07 | 0.01 | 0.14 |
| 2017 | MSZ | 7 | 130 | SC | 0.26 | 64.43 | 18.08 | 8.71 | 1.27 | 0.08 | 0.01 | 0.16 |
| 2017 | MSZ | 7 | 130 | SW | 0.29 | 63.47 | 18.41 | 8.96 | 1.27 | 0.07 | 0.02 | 0.17 |
| 2017 | MSZ | 5 | 130 | SC | 0.29 | 64.80 | 17.63 | 8.68 | 1.27 | 0.07 | 0.01 | 0.16 |
| 2017 | MSZ | 5 | 130 | SW | 0.29 | 63.83 | 18.03 | 9.03 | 1.30 | 0.08 | 0.02 | 0.17 |
| 2017 | MSZ | 2 | 130 | SC | 0.26 | 65.40 | 17.58 | 8.41 | 1.26 | 0.06 | 0.00 | 0.16 |
| 2017 | MSZ | 2 | 130 | SW | 0.30 | 63.98 | 18.36 | 8.52 | 1.25 | 0.07 | 0.00 | 0.18 |
| 2018 | MSZ | 9 | 130 | SC | 0.30 | 66.02 | 17.38 | 7.35 | 1.24 | 0.06 | 0.01 | 0.14 |
| 2018 | MSZ | 9 | 130 | SW | 0.33 | 65.33 | 17.75 | 7.38 | 1.25 | 0.06 | 0.01 | 0.16 |
| 2018 | MSZ | 9 | 130 | SC | 0.30 | 65.82 | 17.75 | 7.25 | 1.24 | 0.06 | 0.00 | 0.13 |
| 2018 | MSZ | 9 | 130 | SW | 0.32 | 65.05 | 18.13 | 7.10 | 1.23 | 0.06 | 0.01 | 0.16 |
| 2018 | MSZ | 2 | 130 | SC | 0.28 | 65.53 | 17.76 | 7.71 | 1.26 | 0.06 | 0.01 | 0.13 |
| 2018 | MSZ | 2 | 130 | SW | 0.30 | 64.98 | 17.81 | 7.96 | 1.26 | 0.06 | 0.02 | 0.14 |
| 2018 | SSZ | 3 | 130 | SC | 0.35 | 60.86 | 19.18 | 10.61 | 1.38 | 0.08 | 0.03 | 0.22 |
| 2018 | SSZ | 3 | 130 | SW | 0.34 | 60.40 | 19.50 | 11.02 | 1.36 | 0.07 | 0.03 | 0.23 |
| 2018 | MSZ | 5 | 130 | SC | 0.31 | 67.58 | 16.58 | 6.93 | 1.23 | 0.06 | 0.00 | 0.11 |
| 2018 | MSZ | 5 | 130 | SW | 0.34 | 66.50 | 17.43 | 6.84 | 1.19 | 0.06 | 0.00 | 0.12 |
| 2018 | MSZ | 9 | 130 | SC | 0.30 | 66.80 | 16.99 | 6.79 | 1.24 | 0.06 | 0.01 | 0.14 |
| 2018 | MSZ | 9 | 130 | SW | 0.33 | 65.89 | 17.58 | 7.02 | 1.23 | 0.06 | 0.02 | 0.12 |
| 2018 | MSZ | 2 | 130 | SC | 0.24 | 65.44 | 17.97 | 7.90 | 1.26 | 0.06 | 0.00 | 0.12 |
| 2018 | MSZ | 2 | 130 | SW | 0.26 | 65.81 | 17.55 | 7.65 | 1.25 | 0.06 | 0.01 | 0.13 |
| | | | Av | SC | 0.28 | 65.17 | 17.68 | 8.20 | 1.27 | 0.07 | 0.01 | 0.15 |
| | | | Av | SW | 0.31 | 64.38 | 18.07 | 8.32 | 1.26 | 0.07 | 0.01 | 0.16 |
| | | | Δ | units | −0.03 | 0.79 | −0.39 | −0.12 | 0.01 | 0.00 | 0.00 | −0.01 |
| | | | Δ | % | −9.68 | 1.23 | −2.16 | −1.44 | 0.79 | 0.00 | 0.00 | −6.25 |

TABLE 2a
seed properties of variety 122 harvested by straight cutting (SC) or swathing (SW). SSZ: Short season zone; MSZ: Mid season zone;
LSZ: Long season zone; DMAT: days to maturity. Av: average across locations; Δ: difference of straight cutting versus swathing.

| | | | | | | Relative | | Oil | | Protein | | | Gluc |
|---|---|---|---|---|---|---|---|---|---|---|---|---|---|
| | | | | | Yield | Yield | | Difference | | Difference | | GLUCOSIN- | Difference |
| Year | Zone | Locations | variety | Treatment | KG/HA | Difference | OIL | (Units) | PROTEIN | (Units) | DMAT | OLATES | (Units) |
| 2017 | LSZ | 1 | 122 | SC | 4.56 | 0.98 | 49.23 | −0.17 | 44.11 | −1.69 | 90.50 | 14.20 | 0.85 |
| 2017 | LSZ | 1 | 122 | SW | 4.64 | | 49.40 | | 45.80 | | 92.50 | 13.35 | |
| 2017 | MSZ | 2 | 122 | SC | 6.50 | 1.23 | 48.20 | 0.80 | 46.91 | 1.32 | 97.00 | 13.59 | 0.28 |
| 2017 | MSZ | 2 | 122 | SW | 5.28 | | 49.00 | | 45.59 | | 96.00 | 13.31 | |
| 2017 | SSZ | 3 | 122 | SC | 5.69 | 1.07 | 50.90 | 0.20 | 40.42 | −0.33 | 101.00 | 15.78 | 1.19 |
| 2017 | SSZ | 3 | 122 | SW | 5.30 | | 50.70 | | 40.75 | | 105.00 | 14.59 | |
| 2017 | SSZ | 4 | 122 | SC | 5.42 | 1.10 | 49.98 | 1.54 | 47.61 | 1.41 | 106.00 | 13.56 | 1.99 |
| 2017 | SSZ | 4 | 122 | SW | 4.91 | | 48.44 | | 46.20 | | 110.00 | 11.57 | |
| 2017 | MSZ | 5 | 122 | SC | 6.47 | 1.12 | 49.30 | 0.25 | 46.64 | 0.42 | 97.00 | 13.36 | 1.74 |
| 2017 | MSZ | 5 | 122 | SW | 5.77 | | 49.05 | | 46.22 | | 97.50 | 11.62 | |
| 2017 | LSZ | 1 | 122 | SC | 4.58 | 1.04 | 49.10 | 0.08 | 44.56 | −1.68 | 90.00 | 12.24 | −0.81 |
| 2017 | LSZ | 1 | 122 | SW | 4.42 | | 49.02 | | 46.24 | | 94.00 | 13.05 | |
| 2017 | MSZ | 2 | 122 | SC | 5.77 | 1.20 | 49.10 | 1.00 | 45.38 | −0.96 | 97.50 | 14.64 | 1.19 |
| 2017 | MSZ | 2 | 122 | SW | 4.79 | | 48.10 | | 46.34 | | 95.50 | 13.45 | |
| 2017 | MSZ | 8 | 122 | SC | 6.44 | 1.06 | 50.31 | −0.16 | 45.86 | −1.17 | 103.50 | 11.28 | −1.21 |
| 2017 | MSZ | 8 | 122 | SW | 6.07 | | 50.47 | | 47.03 | | 103.50 | 12.49 | |
| 2017 | SSZ | 4 | 122 | SC | 5.80 | 1.11 | 49.42 | −0.30 | 47.84 | 1.49 | 108.00 | 10.52 | −2.33 |
| 2017 | SSZ | 4 | 122 | SW | 5.24 | | 49.72 | | 46.35 | | 108.50 | 12.85 | |
| 2018 | LSZ | 1 | 122 | SC | 4.86 | 1.33 | 46.76 | 1.40 | 49.72 | 0.16 | 85.50 | 13.54 | −3.27 |
| 2018 | LSZ | 1 | 122 | SW | 3.65 | | 45.36 | | 49.56 | | 85.50 | 16.81 | |
| 2018 | MSZ | 9 | 122 | SC | 5.05 | 1.11 | 45.90 | 0.10 | 43.98 | −0.95 | 91.00 | 11.53 | 1.10 |
| 2018 | MSZ | 9 | 122 | SW | 4.55 | | 45.80 | | 44.93 | | 91.50 | 10.43 | |
| 2018 | MSZ | 10 | 122 | SC | 4.29 | 1.31 | 47.41 | 1.42 | 48.69 | −0.16 | 96.50 | 13.60 | −2.60 |
| 2018 | MSZ | 10 | 122 | SW | 3.27 | | 45.99 | | 48.85 | | 95.50 | 16.20 | |
| 2018 | LSZ | 1 | 122 | SC | 4.21 | 0.86 | 45.55 | −1.03 | 48.74 | 0.70 | 85.00 | 16.70 | 0.47 |
| 2018 | LSZ | 1 | 122 | SW | 4.88 | | 46.58 | | 48.04 | | 88.00 | 16.23 | |
| 2018 | MSZ | 9 | 122 | SC | 5.04 | 1.23 | 44.45 | 0.30 | 46.71 | −0.20 | 90.50 | 11.39 | −0.32 |
| 2018 | MSZ | 9 | 122 | SW | 4.09 | | 44.15 | | 46.91 | | 91.00 | 11.71 | |
| 2018 | MSZ | 2 | 122 | SC | 5.97 | 1.02 | 48.55 | 0.00 | 48.10 | 0.19 | 91.50 | 13.77 | 1.60 |
| 2018 | MSZ | 2 | 122 | SW | 5.86 | | 48.55 | | 47.91 | | 93.50 | 12.17 | |
| 2018 | SSZ | 3 | 122 | SC | 4.14 | 1.20 | 49.00 | 2.20 | 39.51 | −1.17 | 101.50 | 8.92 | −7.46 |
| 2018 | SSZ | 3 | 122 | SW | 3.46 | | 46.80 | | 40.68 | | 102.50 | 16.38 | |
| 2018 | MSZ | 5 | 122 | SC | 3.05 | 1.03 | 47.50 | 0.80 | 50.26 | 2.70 | 88.50 | 14.14 | 0.19 |
| 2018 | MSZ | 5 | 122 | SW | 2.97 | | 46.70 | | 47.56 | | 89.50 | 13.95 | |
| 2018 | MSZ | 9 | 122 | SC | 4.48 | 1.05 | 45.85 | −0.30 | 44.41 | 0.71 | 89.00 | 18.48 | 2.42 |
| 2018 | MSZ | 9 | 122 | SW | 4.26 | | 46.15 | | 43.70 | | 92.00 | 16.06 | |
| 2018 | MSZ | 2 | 122 | SC | 6.49 | 1.08 | 47.55 | 0.20 | 49.28 | 0.37 | 94.50 | 15.14 | −0.23 |
| 2018 | MSZ | 2 | 122 | SW | 6.03 | | 47.35 | | 48.91 | | 94.50 | 15.37 | |
| 2018 | MSZ | 11 | 122 | SC | 4.38 | 1.56 | 45.20 | 3.23 | 51.17 | 3.02 | 93.50 | 16.93 | 0.56 |
| 2018 | MSZ | 11 | 122 | SW | 2.80 | | 41.97 | | 48.15 | | 95.00 | 16.37 | |
| 2019 | MSZ | 11 | 122 | SC | 4.62 | 1.16 | 49.81 | 3.47 | 42.32 | −0.78 | 115.00 | 14.99 | 1.08 |
| 2019 | MSZ | 11 | 122 | SW | 4.00 | | 46.34 | | 43.10 | | 113.00 | 13.91 | |
| | | | Av | SC | 5.13 | | 48.05 | | 46.30 | | 95.83 | 13.73 | |
| | | | Av | SW | 4.58 | | 47.41 | | 46.13 | | 96.86 | 13.90 | |
| | | | Δ | units | 0.55 | | 0.64 | | 0.17 | | −1.03 | −0.17 | |
| | | | Δ | % | 12.01 | | 1.35 | | 0.37 | | −1.06 | −1.22 | |

TABLE 2b
seed properties of variety 122 harvested by straight cutting (SC) or swathing (SW). SatFAT:
total saturated fatty acids. The fatty acid composition is given in % of oil weight in the
seed. Av: average across locations; Δ: difference of straight cutting versus swathing.

| Year | Zone | Locations | variety | Treatment | SatFAT | C12_0 | C14_0 | C16_0 | C18_0 | C20_0 | C22_0 | C24_0 |
|---|---|---|---|---|---|---|---|---|---|---|---|---|
| 2017 | MSZ | 2 | 122 | SC | 6.36 | 0.01 | 0.04 | 3.78 | 1.56 | 0.54 | 0.28 | 0.16 |
| 2017 | MSZ | 2 | 122 | SW | 6.60 | 0.01 | 0.05 | 3.96 | 1.59 | 0.54 | 0.28 | 0.18 |
| 2017 | SSZ | 3 | 122 | SC | 6.70 | 0.01 | 0.04 | 4.02 | 1.66 | 0.55 | 0.29 | 0.15 |
| 2017 | SSZ | 3 | 122 | SW | 6.94 | 0.01 | 0.04 | 4.15 | 1.70 | 0.59 | 0.31 | 0.15 |
| 2017 | MSZ | 5 | 122 | SC | 6.58 | 0.01 | 0.05 | 3.94 | 1.62 | 0.55 | 0.29 | 0.14 |
| 2017 | MSZ | 5 | 122 | SW | 6.70 | 0.01 | 0.05 | 3.97 | 1.65 | 0.57 | 0.30 | 0.17 |
| 2017 | MSZ | 2 | 122 | SC | 6.41 | 0.01 | 0.05 | 3.82 | 1.57 | 0.55 | 0.27 | 0.16 |
| 2017 | MSZ | 2 | 122 | SW | 6.50 | 0.01 | 0.05 | 3.88 | 1.57 | 0.55 | 0.29 | 0.18 |
| 2018 | MSZ | 9 | 122 | SC | 6.97 | 0.01 | 0.05 | 4.19 | 1.70 | 0.57 | 0.28 | 0.19 |
| 2018 | MSZ | 9 | 122 | SW | 7.15 | 0.01 | 0.05 | 4.30 | 1.72 | 0.59 | 0.29 | 0.21 |
| 2018 | MSZ | 9 | 122 | SC | 7.03 | 0.01 | 0.05 | 4.22 | 1.69 | 0.58 | 0.29 | 0.20 |
| 2018 | MSZ | 9 | 122 | SW | 7.21 | 0.01 | 0.05 | 4.31 | 1.71 | 0.60 | 0.31 | 0.23 |
| 2018 | MSZ | 2 | 122 | SC | 6.72 | 0.01 | 0.05 | 4.02 | 1.66 | 0.54 | 0.28 | 0.17 |
| 2018 | MSZ | 2 | 122 | SW | 6.87 | 0.01 | 0.05 | 4.11 | 1.69 | 0.58 | 0.30 | 0.15 |
| 2018 | SSZ | 3 | 122 | SC | 7.03 | 0.01 | 0.04 | 4.43 | 1.45 | 0.57 | 0.36 | 0.19 |
| 2018 | SSZ | 3 | 122 | SW | 7.09 | 0.01 | 0.04 | 4.46 | 1.45 | 0.58 | 0.37 | 0.20 |
| 2018 | MSZ | 5 | 122 | SC | 6.50 | 0.00 | 0.05 | 3.83 | 1.64 | 0.52 | 0.27 | 0.20 |
| 2018 | MSZ | 5 | 122 | SW | 7.01 | 0.01 | 0.05 | 4.13 | 1.72 | 0.58 | 0.30 | 0.25 |
| 2018 | MSZ | 9 | 122 | SC | 7.00 | 0.01 | 0.05 | 4.17 | 1.73 | 0.58 | 0.28 | 0.19 |
| 2018 | MSZ | 9 | 122 | SW | 7.08 | 0.01 | 0.05 | 4.21 | 1.74 | 0.58 | 0.28 | 0.21 |
| 2018 | MSZ | 2 | 122 | SC | 6.39 | 0.00 | 0.04 | 3.78 | 1.60 | 0.54 | 0.28 | 0.17 |
| 2018 | MSZ | 2 | 122 | SW | 6.66 | 0.00 | 0.04 | 3.97 | 1.69 | 0.58 | 0.30 | 0.22 |
| | | | Av | SC | 6.70 | 0.01 | 0.05 | 4.02 | 1.63 | 0.55 | 0.29 | 0.17 |
| | | | Av | SW | 6.89 | 0.01 | 0.05 | 4.13 | 1.66 | 0.58 | 0.30 | 0.20 |
| | | | Δ | units | −0.19 | 0.00 | 0.00 | −0.11 | −0.03 | −0.03 | −0.01 | −0.03 |
| | | | Δ | % | −2.76 | 0.00 | 0.00 | −2.66 | −1.81 | −5.17 | −3.33 | −15.00 |

TABLE 2c
seed properties of variety 122 harvested by straight cutting (SC) or swathing (SW). The fatty acid composition is given
in % of oil weight in the seed. Av: average across locations; Δ: difference of straight cutting versus swathing.

| | | | | | | | | | | | C22_1 | |
|---|---|---|---|---|---|---|---|---|---|---|---|---|
| Year | Zone | Locations | variety | Treatment | C16_1 | C18_1 | C18_2 | C18_3 | C20_1 | C20_2 | eruc | C24_1 |
| 2017 | MSZ | 2 | 122 | SC | 0.24 | 62.65 | 18.54 | 10.31 | 1.22 | 0.09 | 0.01 | 0.17 |
| 2017 | MSZ | 2 | 122 | SW | 0.27 | 61.84 | 19.00 | 10.46 | 1.15 | 0.06 | 0.00 | 0.20 |
| 2017 | SSZ | 3 | 122 | SC | 0.29 | 62.03 | 18.86 | 10.38 | 1.13 | 0.06 | 0.00 | 0.18 |
| 2017 | SSZ | 3 | 122 | SW | 0.30 | 61.13 | 19.22 | 10.56 | 1.15 | 0.09 | 0.00 | 0.20 |
| 2017 | MSZ | 5 | 122 | SC | 0.28 | 62.21 | 18.73 | 10.36 | 1.19 | 0.07 | 0.01 | 0.18 |
| 2017 | MSZ | 5 | 122 | SW | 0.29 | 61.62 | 19.05 | 10.42 | 1.20 | 0.07 | 0.01 | 0.20 |
| 2017 | MSZ | 2 | 122 | SC | 0.27 | 63.48 | 18.09 | 9.99 | 1.17 | 0.08 | 0.00 | 0.16 |
| 2017 | MSZ | 2 | 122 | SW | 0.28 | 62.15 | 18.90 | 10.41 | 1.19 | 0.07 | 0.00 | 0.16 |
| 2018 | MSZ | 9 | 122 | SC | 0.29 | 63.45 | 18.33 | 9.13 | 1.18 | 0.06 | 0.01 | 0.14 |
| 2018 | MSZ | 9 | 122 | SW | 0.30 | 62.63 | 18.84 | 9.19 | 1.19 | 0.06 | 0.01 | 0.17 |
| 2018 | MSZ | 9 | 122 | SC | 0.29 | 62.88 | 18.88 | 9.04 | 1.20 | 0.06 | 0.02 | 0.16 |
| 2018 | MSZ | 9 | 122 | SW | 0.32 | 62.55 | 19.26 | 8.72 | 1.19 | 0.07 | 0.02 | 0.18 |
| 2018 | MSZ | 2 | 122 | SC | 0.28 | 64.01 | 18.38 | 8.77 | 1.19 | 0.07 | 0.02 | 0.15 |
| 2018 | MSZ | 2 | 122 | SW | 0.29 | 63.39 | 18.38 | 9.20 | 1.21 | 0.06 | 0.02 | 0.15 |
| 2018 | SSZ | 3 | 122 | SC | 0.36 | 58.71 | 19.66 | 12.09 | 1.31 | 0.08 | 0.03 | 0.26 |
| 2018 | SSZ | 3 | 122 | SW | 0.35 | 57.94 | 19.99 | 12.37 | 1.34 | 0.08 | 0.03 | 0.28 |
| 2018 | MSZ | 5 | 122 | SC | 0.30 | 65.72 | 17.48 | 8.22 | 1.18 | 0.05 | 0.01 | 0.13 |
| 2018 | MSZ | 5 | 122 | SW | 0.35 | 63.97 | 18.55 | 8.22 | 1.17 | 0.06 | 0.01 | 0.15 |
| 2018 | MSZ | 6 | 122 | SC | 0.29 | 64.09 | 18.14 | 8.63 | 1.18 | 0.06 | 0.01 | 0.15 |
| 2018 | MSZ | 9 | 122 | SW | 0.30 | 63.60 | 18.41 | 8.74 | 1.18 | 0.06 | 0.02 | 0.15 |
| 2018 | MSZ | 2 | 122 | SC | 0.24 | 64.63 | 17.96 | 9.06 | 1.20 | 0.06 | 0.00 | 0.13 |
| 2018 | MSZ | 2 | 122 | SW | 0.26 | 63.86 | 18.30 | 9.05 | 1.19 | 0.06 | 0.00 | 0.14 |
| | | | Av | SC | 0.28 | 63.08 | 18.46 | 9.63 | 1.20 | 0.07 | 0.01 | 0.16 |
| | | | Av | SW | 0.30 | 62.24 | 18.90 | 9.76 | 1.20 | 0.07 | 0.01 | 0.18 |
| | | | Δ | units | −0.02 | 0.84 | −0.44 | −0.13 | 0.00 | 0.00 | 0.00 | −0.02 |
| | | | Δ | % | −6.67 | 1.35 | −2.33 | −1.33 | 0.00 | 0.00 | 0.00 | −11.11 |

It can be seen from Tables 1 and 2 that straight cutting increased the seed yield in both varieties with about 12%. This increase in seed yield was consistently seen across locations.

There is a trend to an increased oil as well as an increased protein content, which was observed for both varieties. There is also a trend towards a reduction of the number of days to maturity both in the earlier maturing and in the later maturing variety.

Furthermore, both varieties showed a trend in reduction of the levels of glucosinolates.

Unexpectedly, the levels of saturated fatty acids consistently decreased consistently across locations and over the years in both varieties. The levels of C12:0 and C14:0 were too low to measure differences between straight cut and swathing, but all other different saturated fatty acids (C16:0 up to C24:0) contributed similarly to this reduction in total saturated fatty acids.

There was also a consistent increase in the levels of C18:1, and a consistent reduction in the levels of C16:1, C18:2, C18:3, and C24:1.

Thus, surprisingly, the podshatter resistance trait, through its ability to allow the plants to grow to full maturity, does not only increase the seed yield, but also has beneficial effects of the oil quality, and in particular on the levels of saturated fatty acids.

In conclusion, embodiments according to the invention are summarized in the following paragraphs:
Paragraph 1. A method for enhancing oil characteristics in a Brassica oilseed plant, said method comprising growing Brassica oilseed plants, and harvesting the seeds by straight cutting.
Paragraph 2. The method of paragraph 1, wherein said Brassica oilseed plants are podshatter resistant.
Paragraph 3. The method of paragraph 2, wherein the Brassica oilseed plant contains a modified Indehiscent gene.
Paragraph 4. The method of paragraph 1, wherein podshattering is inhibited by application of pod sealants to the growing Brassica oilseed plants.
Paragraph 5. The method of any one of paragraphs 1 to 4, which is a method to increase oil quantity.
Paragraph 6. The method of any one of paragraphs 1 to 4, which is a method to reduce the levels of saturated fatty acids in the oil.
Paragraph 7. The method of paragraph 6, which is a method to increase oil healthiness.
Paragraph 8. The method of any one of paragraphs 1 to 7, wherein said Brassica oilseed plant is Brassica napus.
Paragraph 9. The method of paragraph 8, wherein said Brassica napus plant is a hybrid.
Paragraph 10. The method according to any one of paragraphs 1 to 9, wherein said Brassica oilseed plant is resistant to a herbicide.
Paragraph 11. The method according to paragraph 10, further comprising treating the growing Brassica oilseed plants with a herbicide.
Paragraph 12. A method of producing Brassica oilseed oil with enhanced characteristics, said method comprising growing Brassica oilseed plants, harvesting the seeds by straight cutting, and extracting the oil from said seeds.
Paragraph 13. The method of paragraph 12, wherein the enhanced characteristic is improved health.
Paragraph 14. Use of the seed obtained using the method of any one of paragraphs 1-11 for the production of oil with enhanced characteristics.
Paragraph 15. Use of the oil obtained using the method of paragraph 13 as food ingredient.

1. A method for enhancing oil characteristics in a Brassica oilseed plant, said method comprising growing Brassica oilseed plants, and harvesting seeds from said plants by straight cutting.

2. The method of claim 1, wherein said Brassica oilseed plants are podshatter resistant.

3. The method of claim 2, wherein the Brassica oilseed plant contains a modified Indehiscent gene.

4. The method of claim 1, wherein podshattering is inhibited by application of pod sealants to the growing Brassica oilseed plants.

5. The method of claim 1, which is a method to increase oil quantity.

6. The method of claim 1, which is a method to reduce the levels of saturated fatty acids in the oil.

7. The method of claim 6, which is a method to increase oil healthiness.

8. The method of claim 1, wherein said Brassica oilseed plant is Brassica napus.

9. The method of claim 8, wherein said Brassica napus plant is a hybrid.

10. The method according to claim 1, wherein said Brassica oilseed plant is resistant to a herbicide.

11. The method according to claim 10, further comprising treating the growing Brassica oilseed plants with a herbicide.

12. A method of producing Brassica oilseed oil with enhanced characteristics, said method comprising growing Brassica oilseed plants, harvesting seeds from said plants by straight cutting, and extracting the oil from said seeds.

13. The method of claim 12, wherein the enhanced characteristic is improved health.

14. (canceled)

15. A food product comprising the oil obtained using the method of claim 13.
